(19) United States
(12) Patent Application Publication
(10) Pub. No.: US 2009/0101060 A1
(43) Pub. Date: Apr. 23, 2009

(54) Dip and see sugar indicator

(76) Inventor: Dilcia M. Granville, Elmont, NY (US)

Correspondence Address: Dilcia M. Granville, 1623 Stuyvesant St., Elmont, NY, 11003, US

(21) Appl. No.: 12/221,076

(22) Filed: Jul. 30, 2008

Related U.S. Application Data
(60) Provisional application No. 60/955,546, filed on Sep. 10, 2007.

(51) Int. Cl. G01N 33/02

(52) U.S. Cl. 116/206

(57) ABSTRACT

A Dip and See spoon shape strips detect sugar in food. The sugar tester when briefly dip in the food, it indicates whether or not the product contain sugar. The indicator is impregnated with components and blue background dye. The oxygen liberated in the final reaction binds with dye to produce a series of color changes 30 seconds after wetting the spoon shape strip with sugar solution. Color changes after wetting the strips from blue-green to greenish brown in sugar solution. The tester will present a variation shade of brown. Lighter shade of brown means a small concentration of sugar and darker shade of brown means large concentration of sugar in the food. If strip maintains blue-green color, after wetting it, the product contains no sugar. Results are obtainable by direct comparison of the test strip with the color blocks printed on the instructions. No calculations or laboratory instruments are required. The tester is a quick, safe, reliable and is disposable.

### BACKGROUND OF THE INVENTION

1. Field of Invention

The present invention relates generally to sugar indicator, spoon shape strip and more specifically it relates to a Dip and See for detecting sugar in food. Interest in detecting sugar in food is high and growing as consumers increasingly try to avoid sugar due to health concerns such as people suffering from diabetes, obesity and wanting to change lifestyle.

2. Description Related to Art

It can be appreciated that sugar indicator strip have been use for years. Typically, sugar indicator strips are comprised of products designed to measure sugar in human blood. This new product is unique because it could detect the concentration of sugar in food products in an aesthetic, reliable, quick and safe manner.

The main problem with conventional sugar indicator strips is that they can only detect glucose in the human blood.

While these devices may be suitable for particular purpose to which they address, they are not as suitable for detecting sugar in food. Interest in detecting sugar in food is high and growing as consumers increasingly try to avoid sugar due to health concerns such as people suffering from diabetes, obesity and wanting to change lifestyle.

In these respects, the dip and see sugar indicator according to the present invention substantially departs from the conventional concepts and designs of the prior art, and in so doing, provides an apparatus primarily developed for the purpose of detecting concentration of sugar in food. Users will be able to quickly observe whether or not their food contains sugar and their concentration. The art changes color if the food contains sugar and remains the same if the food is sugar free. This is a useful art for the millions of American who are at risk of Type II diabetes and want to control their sugar intake. The art is also useful for the other millions who are concerned about healthy eating and positive lifestyle.

### SUMMARY OF INVENTION

In view of the foregoing disadvantages inherent in the known types of sugar indicator strip now present in the prior art, the present invention provides a new dip and see sugar tester construction wherein the same can be utilized for detecting sugar in food. The present invention is a quick, reliable, safe and allow the users to know the concentration of sugar in their food. Interest in detecting sugar in food is high and growing as consumers increasingly try to avoid sugar due to health concerns such as people suffering from diabetes, obesity and wanting to change lifestyle.

The general purpose of the present invention, which will be described subsequently in greater detail, is to provide a new dip and see that has many of the advantages of the sugar indicator strip mentioned heretofore and many novel features that result in a new dip and see which is not anticipated, rendered obvious, suggested, or even implied by any of the prior art sugar indicator strip, either alone or in any combination thereof.

To attain this, the present invention generally comprises 16.3% w/w/Glucose Oxidase (Aspergillus Niger, 1.3 IU) 0.6% w/w/ Peroxide (Horseradish 3300 IU) 7.0% w/w/ Potassium iodide 76.1% w/w Buffer). The indicator spoon is impregnated with the components listed above and a blue background dye. The oxygen liberated in the final reaction binds with dye to produce a series of color changes 30 seconds after wetting the spoon with sugar solution. The tester will present a variation shade of brown. Lighter shade of brown means a small concentration of sugar and darker shade of brown means large concentration of sugar in the food. If strip maintains blue-green color, after wetting it, the product contains no sugar. Results are obtainable by direct comparison of the test strip with the color blocks printed on the instructions. No calculations or laboratory instruments are required. The tester is a quick, safe, reliable and is disposable.

There has thus been outlined, rather broadly, the more important features of the invention in order that the detailed description thereof may be understood, and in order that the present contribution to the art may be better appreciated. There are additional features of the invention that will be described hereinafter.

In this respect, before explaining at least one embodiment of the invention in detail, it is to be understood that the invention is not limited in its application to the details of the construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a Dip and See sugar indicator that will overcome the shortcomings of the prior art devices.

An object of the present invention is to provide a dip and see for detecting sugar in food in safe, accurate, easy and reliable method for people who must avoid sugar due to health concerns such as people suffering from diabetes, obesity and wanting to change their lifestyles.

Another object is to provide a dip and see sugar detector prior to consumption of the food. Sugar is added in many products that people do not normally assume contain sugar. The amount found in beverage sold can actually be quite high. Having a reliable quick indicator could prove very important for many people, especially those who are very sensitive to sugar and those with health problems such as bariatric, pediatric, diabetes obese patients, and for weight control where sugar avoidance is particularly beneficial.

Another object is that the tester will present a variation shade of brown. Lighter shade of brown means a small concentration of sugar and darker shade of brown means large concentration of sugar in the food. If strip maintains blue-green color, after wetting it, the product contains no sugar. Results are obtainable by direct comparison of the test strip with the color blocks printed on the instructions. No calculations or laboratory instruments are required. The tester is aesthetic, quick, safe, and reliable and is disposable.

Another object is to provide a dip and see sugar indicator that provide people with choices and control. When people dine out, travel and when they want to make sure that the beverage they ordered contain no sugar. Many times, self serve beverages, ice creams found in fast food stores, restaurants' self serve fountain beverages contain a great deal of sugar, even though they are labeled to be sugar free.

Another object is to provide to parent's of pediatric patients with a tool. Parents who have children suffering from diabetes are very concerned about the children's beverage intakes. Many times parents taste their children's beverages, prior to let their children consume it. This method is not always reliable as parents complain afterwards, that the product they thought to be sugar free contained sugar. Dip and See sugar indicator is safe for children to test their own beverage. Dip and See provides independence and control for the children as well for their parents. The art is compact, easy to carry and could be attached to a key chain. It is attractive and socially acceptable, everyone could use without feeling stigmatized.

Another object is to provide a sugar indicator that creates awareness not only to adults but also to children about taking control of their diet by becoming aware of their sugar intake.

Another object is to provide a sugar indicator that may lead to a reduction in demand for products containing sugar and that can only benefit health and society as a whole.

Other objects and advantages of the present invention will become obvious to the reader and it is intended that these objects and advantages are within the scope of the present invention.

### BRIEF DESCRIPTION OF THE DRAWINGS

Various other objects, features and attendant advantages of the present invention will become fully appreciated as the same becomes better understood when considered in conjunction with the accompanying drawing in which like reference characters designate the same or similar parts throughout, and wherein:

FIG. 1 is a plastic test strips in a shape of spoon impregnated with components and a blue background dye.

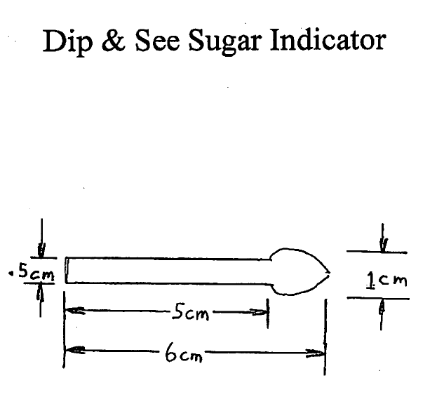

### DETAILED DESCRIPTION OF THE INVENTION

Turning now descriptively to the drawing, in which similar reference characters denote similar elements throughout the several views, the attached FIGURE illustrate a Dip and See sugar indicator, spoon shape strip, which comprises 16.3% w/w/Glucose Oxidase (Aspergillus Niger, 1.3 IU) 0.6% w/w/ Peroxide (Horseradish 3300 IU) 7.0% w/w/ Potassium Iodide 76.1% w/w Buffer). The indicator spoon is impregnated with the components listed above and a blue background dye. The oxygen liberated in the final reaction binds with dye to produce a series of color changes 30 seconds after wetting the strip with sugar solution.

One enzyme, glucose oxidase, catalyzes the formation of gluconic acid and hydrogen peroxide, from the oxidation of glucose. A second enzyme, peroxide, catalyzes the reaction of hydrogen peroxide with potassium iodide chromogen to oxidize the chromogen to colors ranging from blue-green to greenish-brown through brown and dark brown. The indicator must be stored at temperature between 59-86 degree Fahrenheit and out of direct light.

The spoon shape strip is impregnated with the components listed and a blue background dye. The oxygen liberated in the final reaction binds with the dye to produce a series of color changes 30 seconds after wetting the strip with sugar solution.

Dip and See sugar indicators are disposable, firm plastic strips for which several reagent components are impregnated and work by 1) Completely emerging reagent areas of the trip in the liquid, soft food, puree, for 30 seconds. 2) While removing, touch the side of the strip against the rim of the beverage to remove excess liquid. 3) See color changes after wetting the strips from blue-green to greenish brown. 4) If strip maintains blue-green color, after wetting it, the product contains no sugar. 5) Results are obtainable by direct comparison of the test strip with the color blocks printed on the instructions. No calculations or laboratory instruments are required.

As to further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur too those skilled in the art, it is not desired to limit the invention to the exact construction and operation shown and described, and accordingly, all suitable modifications and equivalents may be resorted to, falling within the scope of the invention.

What is claimed is:

1. A plastic spoon shaped indicator adapted to inform the user of the presence of sugar in food, specifically drinks, in safe, accurate, easy and reliable method for people who must avoid sugar due to health concerns. Dip and See sugar indicators are disposable.

2. The Dip and See sugar indicator will present a shade of brown, meaning that a lighter shade of brown means a small concentration of sugar and a darker shade of brown means a large concentration of sugar in food.

3. No calculations or laboratory instruments are required. The tester is aesthetic, quick, safe, and reliable and is disposable.

4. The Dip and See sugar indicator that may lead to a reduction in demand for products containing sugar which can benefit the health of society as a whole.
